(19) United States
(12) Patent Application Publication
(10) Pub. No.: US 2025/0205481 A1
(43) Pub. Date: Jun. 26, 2025

(54) SYSTEMS AND METHODS OF USING PULSED ELECTRIC FIELDS TO SPATIALLY CONTROL CELL DEATH MECHANISMS THAT MODULATE THE IMMUNE RESPONSE

(71) Applicant: Virginia Tech Intellectual Properties, Inc., Blacksburg, VA (US)

(72) Inventors: Rafael V. Davalos, Aptos, CA (US); Kenneth N. Aycock, Blacksburg, VA (US); Nastaran Alinezhadbalalami, Blacksburg, VA (US); Irving Coy Allen, Blacksburg, VA (US)

(21) Appl. No.: 18/846,198

(22) PCT Filed: Mar. 13, 2023
(86) PCT No.: PCT/US23/15118

§ 371 Date: Sep. 11, 2024

Related U.S. Application Data
(60) Provisional application No. 63/318,996, filed on Mar. 11, 2022.
(60) Provisional application No. 63/323,098, filed on Mar. 24, 2022.
(60) Provisional application No. 63/414,773, filed on Oct. 10, 2022.

(51) Int. Cl. A61N 1/36; A61N 1/02; A61N 1/05; A61N 1/08; A61N 1/32

(52) U.S. Cl. CPC A61N 1/36002; A61N 1/025; A61N 1/08; A61N 1/05; A61N 1/327

(57) ABSTRACT

The present invention provides systems and methods for treatment planning and/or administering electrical pulses that elicit desired cell death mechanisms. Embodiments of the invention include selection of pulse parameters to tailor cell death type.

### CROSS-REFERENCE TO RELATED APPLICATIONS

This application relies on the disclosure of and claims priority to and the benefit of the filing date of U.S. Provisional Application Nos. 63/414,773 filed Oct. 10, 2022, 63/323,098 filed Mar. 24, 2022, and 63/318,996 filed Mar. 11, 2022, which applications are hereby incorporated by reference herein in their entireties.

### BACKGROUND OF THE INVENTION

Irreversible electroporation (IRE) and high frequency irreversible electroporation (HFIRE) are tissue ablation strategies used in clinical and preclinical settings to treat aggressive tumors such as pancreatic, liver, and brain tumors with promising outcomes (Tameez Ud Din, A. et al., “Irreversible Electroporation for Liver Tumors: A Review of Literature”, Cureus 11, 2019; Martin, R. C. G., “Use of irreversible electroporation in unresectable pancreatic cancer”, Hepatobiliary Surg. Nutr. 4, 211-215, 2015; Garcia, P. A. et al., “Intracranial nonthermal irreversible electroporation: In vivo analysis”, J. Membr. Biol. 236, 127-136, 2010). Employing brief (1-100 μs), high amplitude (1-5 kV/cm) pulsed electric fields (PEFs), IRE and HFIRE generate nanoscale defects in targeted cell membranes to treat tumors. HFIRE was developed as a second-generation technology to mitigate the undesirable side effects of IRE such as muscle contractions (Arena, C. B. et al., “High-frequency irreversible electroporation (H-FIRE) for non-thermal ablation without muscle contraction”, Biomed. Eng. Online 10, 1-20, 2011).

The applied pulses in both treatment modalities disrupt cellular homeostasis to such an extent that cells cannot recover. Cell death occurs on the order of a few minutes to several hours depending on the applied pulse parameters, the targeted cell type, and electric field magnitude. This is related to the type of cell death elicited by the pulses in each location relative to the electrodes, discussed in detail below.

Seemingly due to its non-thermal nature, IRE and HFIRE have been shown to stimulate release of large quantities of antigens from treated cells, allowing for more potent antigen presentation and consequently activation of T cells in comparison to other focal ablation strategies (Shao, Q. et al., “Engineering T cell response to cancer antigens by choice of focal therapeutic conditions”, Int. J. Hyperth. 36, 130-138, 2019). These antigens may exhibit preserved integrity and orientation compared to those released by ablation with other modalities, enhancing their immunogenicity. It has also been demonstrated that manipulations to the voltage waveform can generate different outcomes in terms of cell death (Mercadal, B. et al., “Dynamics of Cell Death After Conventional IRE and H-FIRE Treatments”, Ann. Biomed. Eng. 48, 1451-1462, 2020; Wasson, E. M. et al. “Understanding the role of calcium-mediated cell death in high-frequency irreversible electroporation”, Bioelectrochemistry 131, 2020) and T cell activation (Alinezhadbalalami, N. et al., “Generation of Tumor-activated T cells Using Electroporation”, Bioelectrochemistry 142, 107886, 2021). In particular, high-frequency bipolar pulses (H-FIRE)—have been shown to generate a higher degree of delayed cell death (Mercadal, 2020), as well as more effective activation of cytotoxic CD8+ T cells (Alinezhadbalalami, 2021).

Depending on the previously mentioned factors, IRE and HFIRE can generate either accidental cell death (ACD) or regulated cell death (RCD)—or even a distribution of both within the vicinity of the electrodes. The most widely known form of ACD is necrosis—sometimes called immediate cell death—where affected cells lose membrane integrity, releasing large quantities of damage-associated molecular patterns (DAMPs) as they quickly lose viability. On the other hand, apoptosis—a “quiet” cell death mechanism—involves several highly programmed signaling cascades and is generally considered non-immunogenic. Cells undergo apoptosis as a part of general tissue maintenance, especially for tissues with high cellular turnover such as the intestinal epithelium. Other forms of RCD have recently been identified, two of which are commonly reported in PEF applications, namely necroptosis (Wasson, 2020; López-Alonso, B. et al., “Histopathological and Ultrastructural Changes after Electroporation in Pig Liver Using Parallel-Plate Electrodes and High-Performance Generator”, Sci. Rep. 9, 2019) and pyroptosis (Ringel-Scaia, V. M. et al., “High-frequency irreversible electroporation is an effective tumor ablation strategy that induces immunologic cell death and promotes systemic anti-tumor immunity”, EBioMedicine 44, 112-125, 2019; Zhang, Y. et al., “Molecular and histological study on the effects of non-thermal irreversible electroporation on the liver”, Biochem. Biophys. Res. Commun. 500, 665-670, 2018). Necroptosis is an inflammatory programed form of cell death that is morphologically similar to necrosis. Pyroptosis on the other hand, is a form of RCD that is mediated by proinflammatory caspase activation pathways (CASP1) and is morphologically distinct from apoptosis (Tang, D. et al., “The molecular machinery of regulated cell death”, Cell Res. 29, 347-364, 2019). One or more types of cell death can occur especially in complex biological systems. Additionally, there are specific biochemical or biological processes associated with each type of cell death that can be evaluated to define the type of cell death. Due to the inflammatory nature of both cell death mechanisms, they are considered favorable forms of cell death when an immune response is advantageous.

Recently an immune response following IRE and HFIRE treatments has been identified. It has been shown that IRE treatments induce an adaptive immune response that is hypothesized to be dependent on the cytotoxic T lymphocytes. The immune response has been shown to be improved when used in combination with immune checkpoint blockades (Zhao, J. et al., “Irreversible electroporation reverses resistance to immune checkpoint blockade in pancreatic cancer”, Nat. Commun. 10, 899, 2019).

It has also been shown that cytotoxic T cells can be activated with HFIRE treatments. The extent of the T cell activation seemed to be dependent on the pulsing protocol (Alinezhadbalalami, 2021) alluding to the idea that the level of inflammation can vary changing the pulse parameters. It has also been suggested that the type of cell death can vary based on the applied pulsing protocol, showing a shift in cell death mechanism with pulse widths that are 10 microseconds long in comparison to bursts with shorter pulse durations (Wasson, 2020).

With conventional IRE, the degree to which the tissue conductivity changes during treatment has been suggested to correlate with the reduction of regulatory T cells (Tregs) in the tumor microenvironment (Beitel-White, N. et al., “Real-time prediction of patient immune cell modulation during irreversible electroporation therapy”, Sci. Rep. 9, 17739, 2019). It is well known that pulse parameters can be tuned to modulate the degree of pore formation spatially, changing local tissue conductivity.

Though it is well agreed upon that cell death following IRE is the result of long-lived chemical imbalances across the membrane, the exact physical means by which cells die are not well understood. Though a certain portion of the ablated region is directly caused by a disturbance in homeostatic equilibrium related to the presence of long-lived pores within the cellular membrane, other mechanisms of cellular injury also occur because of the induced field and applied current. A brief summary of the current understanding of IRE-induced cell death is given below.

Due to the strong electric field gradient created by needle electrodes, vastly different effects are likely contributing to cell death depending upon cellular location relative to the electrodes. For instance, close to the electrodes (i.e. ≤˜3 mm), electric fields in excess of 3 kV/cm are common, which are likely associated with Joule heating, and potentially also transient pressure waves, which could stimulate cell death through thermal and mechanical cues. Further from the electrodes (i.e. ˜3-10 mm), the presence of moderate electric fields capable of inducing IRE could lead to electro-conformational denaturation of proteins and macromolecules (Aycock, K. N. & Davalos, R. V., “Irreversible Electroporation: Background, Theory, and Review of Recent Developments in Clinical Oncology”, Bioelectricity 1, 214-234, 2019). Along with this, adenosine triphosphate (ATP) depletion, electrochemical effects, and even vascular disruptions, could all be contributing to cellular injury. Finally, in the more distal areas (i.e. ≥10 mm), reversible electroporation occurs, which could be accompanied by uptake of Ca²′ and/or other electrolytic products. Importantly, it is well understood that a multitude of biophysical and electrochemical effects are generated by application of IRE and other electroporation-based modalities, and in a given treatment a subset of these effects collectively contributes to cell death depending upon the tissue type, electrode geometry, and pulsing parameters.

From an immunological perspective, the degree of adaptive immunity to certain antigens, or groups of antigens, is assumed to be proportional to the innate immune response to that antigen. The four major types of cell death: apoptosis, necrosis, necroptosis, and pyroptosis, are all thought to release different degrees of DAMPs, which play a major role in their immunogenicity.

Recent work has also shown that IRE/HFIRE is effective in inducing systemic anti-tumor immune responses (Ringel-Scaia, 2019; Goswami, I. et al., “Irreversible electroporation inhibits pro-cancer inflammatory signaling in triple negative breast cancer cells”, Bioelectrochemistry, 2017; 113:42-50; Felsted, A. et al., “Histotripsy Ablation Stimulates Potentially Therapeutic Tumor-directed Systemic Immunity”, Society of Interventional Oncology; Boston MA, 2019; Hendricks, A. et al., “Histotripsy initiates local and systemic immunological response and reduces tumor burden in breast cancer”, The Journal of Immunology, 2019, 202; Hendricks, A. et al., “Investigation of the local and systemic immune response to histotripsy ablation of breast cancer in a mouse model”, International Society of Therapeutic Ultrasound; Barcelona, Spain, 2019; Worlikar, T. et al., “Non-Invasive Orthotopic Liver Tumor Ablation Using Histotripsy in an in Vivo Rodent Hepatocellular Carcinoma (HCC) Model”, Society of Interventional Oncology, Boston, MA, 2019), resulting in post-treatment tumor regression both locally and at metastatic sites.

A need remains for methods of selecting specific treatment parameters (i.e. pulse duration, interphase delay, interpulse delay, polarity, delivery rate, energized time, number of bursts, etc.), electrode configurations (i.e. monopolar probe(s) with or without a grounding pad, bipolar probe(s), flat plate electrodes, etc.), adjuvant molecules (i.e. sucrose, calcium, saline, chemotherapeutics, etc.), and probe designs (i.e. internally cooled/heated, integrated phase change materials, etc.) that allow for control of the biophysical, mechanical, and electrochemical effects within different regions relative to the electrode(s), allowing for modulation of the type of cell death elicited by the treatment in different regions. This approach will govern the downstream innate and adaptive immune response. The magnitude and temporal nature of the induced immune response is expected to play a critical role in the outcome of each patient.

### SUMMARY OF THE INVENTION

Aspects of the invention include Aspect 1, which is a method of treating tissue with electrical pulses comprising administering a plurality of electrical pulses to a tissue according to a pulsing protocol expected to achieve a desired cell death gradient.

Aspect 2 is the method of Aspect 1, wherein the desired cell death gradient comprises multiple types of cell death mechanisms.

Aspect 3 is the method of Aspect 1 or 2, wherein the cell death gradient is capable of shifting a local tumor microenvironment from anti-inflammatory to pro-inflammatory, or from pro-inflammatory to anti-inflammatory, in response to the administering of the plurality of electrical pulses.

Aspect 4 is the method of any of Aspects 1-3, wherein the cell death gradient is capable of activating a local innate immune response and a systemic anti-tumor adaptive immune response, reducing metastatic lesions and/or preventing recurrence in tumors, such as mammary tumors, such as a predetermined immune response.

Aspect 5 is a method of treating tissue comprising: determining a desired gradient of cell death within a tissue; constructing a pulsing protocol capable of achieving the desired gradient; and applying a plurality of electrical pulses to tissue according to the pulsing protocol.

Aspect 6 is the method of any of Aspects 1-5, wherein the plurality of electrical pulses is selected to elicit apoptosis, pyroptosis, necroptosis, necrosis, programmed necrosis, and/or coagulative necrosis according to the desired gradient of cell death.

Aspect 7 is the method of any of Aspects 1-6, further comprising monitoring: treatment progression; one or more effect of the plurality of electrical pulses on the tissue; the extent of one or more of the cell death(s) elicited; any change in tissue Joule heating; any change in temperature; any change in impedance (such as determined by FAST impedance measurements); cellular integrity or cellular recovery; the extent of non-thermal or thermal damage, if any; any thermal effects; and/or potential for Joule heating or thermal damage.

Aspect 8 is the method of any of Aspects 1-7, further comprising adjusting one or more parameter of the pulsing protocol to change the gradient and/or extent of cell death, such as in response to the monitoring.

Aspect 9 is the method of any of Aspects 1-8, wherein the change in the gradient of cell death is a change in the extent or degree of apoptosis, pyroptosis, necroptosis, necrosis and/or coagulative necrosis.

Aspect 10 is the method of any of Aspects 1-9, wherein the constructing of the pulsing protocol is performed by reference to a model.

Aspect 11 is a method of treating tissue with electrical pulses comprising: administering a plurality of electrical pulses to a tissue; wherein the plurality of electrical pulses is capable of causing apoptosis, pyroptosis, necroptosis, necrosis and/or coagulative necrosis in selected zones of the tissue.

Aspect 12 is a method for treating tissue comprising: placing a probe in or near tissue within a body, such as a mammal or human, wherein the probe has at least a first electrode; applying a plurality of electrical pulses with the first electrode and optionally one or more additional electrodes, such as a second electrode; causing irreversible electroporation (IRE) of the tissue within a target ablation zone; wherein the electrical pulses are applied to the tissue such that a desired cell death mechanism occurs to stimulate or modulate an immune system response to the tissue, such as a predetermined immune response; optionally further administering one or more adjuvant, such as within the target ablation zone, to achieve a desired/predetermined cell death volume and/or immune system response.

Aspect 13 is the method of any of Aspects 1-12, desired cell death mechanism is chosen from coagulative necrosis, programmed necrosis, necrosis, necroptosis, pyroptosis, and apoptosis.

Aspect 14 is a treatment planning method comprising: selecting one or more desired cell death mechanism; receiving and processing information from medical images of a target tissue and preparing a reconstruction model of the target tissue; using the model of the target tissue, one or more patient-specific characteristics, and one or more of the desired cell death mechanisms, constructing an electroporation protocol capable of achieving one or more of the desired cell death mechanisms in the target tissue and/or a desired gradient/pattern thereof.

Aspect 15 is a method of determining one or more cell death mechanism for an electroporation treatment protocol, comprising: receiving and processing information from medical images of a tissue and preparing a reconstruction model of the tissue; using the model of the tissue and one or more electroporation treatment pulse parameter, generating an image of one or more electroporation zones expected from applying one or more of the treatment pulse parameters to the tissue; and determining an expected cell death mechanism associated with one or more of the electroporation zones.

Aspect 16 is the method of any of Aspects 1-15, further comprising adjusting the electroporation protocol or one or more of the electroporation treatment pulse parameters to change the expected cell death mechanism to another cell death mechanism, such as coagulative necrosis, programmed necrosis, necrosis, necroptosis, pyroptosis, or apoptosis.

Aspect 17 is the method of any of Aspects 1-16, further comprising applying a plurality of electrical pulses to the tissue to administer the electroporation protocol or the electroporation treatment pulse parameters and to elicit the expected cell death mechanism in one or more or each of the electroporation zones.

Aspect 18 is the method of any of Aspects 1-17, further comprising including in the determining of the expected cell death mechanism(s) an expected effect of administration of one or more type of adjuvants.

Aspect 19 is the method of any of Aspects 1-18, further comprising including in the determining of the expected cell death mechanism(s) an expected effect of electrode cooling.

Aspect 20 is the method of any of Aspects 1-19, wherein the electroporation protocol or the one or more electroporation treatment pulse parameter comprises a parameter chosen from pulse width, voltage, polarity, interpulse delay, interphase delay, frequency, number of pulses, and total energized time.

Aspect 21 is a method of activating an immune response to electroporation comprising: placing one or more electrode within a body, such as a mammal or human body; applying a plurality of electrical pulses through the one or more electrode; and causing electroporation of cells within a treatment area, such that the treatment area comprises one or more electroporation zones defined by a specific type of cell death capable of occurring in that electroporation zone; wherein the electroporation is capable of activating an immune response, such as a predetermined immune response; optionally wherein: cells in a first electroporation zone are killed via a first cell death mechanism; cells in a second electroporation zone are killed via a second cell death mechanism; cells in a third electroporation zone are killed via a third cell death mechanism; cells in a fourth electroporation zone are killed via a fourth cell death mechanism; and/or cells in a fifth electroporation zone are killed via a fifth cell death mechanism.

Aspect 22 is the method of any of Aspects 1-21, wherein at least 50% of the cells in the first electroporation zone are killed via the first cell death mechanism; and/or wherein at least 50% of the cells in the second electroporation zone are killed via the second cell death mechanism; and/or wherein at least 50% of the cells in the third electroporation zone are killed via the third cell death mechanism; and/or wherein at least 50% of the cells in the fourth electroporation zone are killed via the fourth cell death mechanism; and/or wherein at least 50% of the cells in the fifth electroporation zone are killed via the fifth cell death mechanism.

Aspect 23 is the method of any of Aspects 1-22, wherein the first, second, third, fourth and/or fifth cell death mechanism(s) is chosen from coagulative necrosis, programmed necrosis, necrosis, necroptosis, pyroptosis, and apoptosis.

Aspect 24 is the method of any of Aspects 1-23, further comprising a sixth electroporation zone, wherein cells are reversibly electroporated.

Aspect 25 is the method of any of Aspects 1-24, wherein the first electroporation zone is located closest to the one or more electrode and the sixth electroporation zone is located furthest from the one or more electrode.

Aspect 26 is the method of any of Aspects 1-25, wherein the plurality of electrical pulses is applied such that the volume of the first electroporation zone is smaller than any other zone.

Aspect 27 is the method of any of Aspects 1-26, wherein the plurality of electrical pulses is applied such that the first cell death mechanism is coagulative necrosis or necrosis.

Aspect 28 is the method of any of Aspects 1-27, wherein the one or more electrode is cooled during the applying.

Aspect 29 is the method of any of Aspects 1-28, further comprising administering one or more adjuvant.

Aspect 30 is the method of any of Aspects 1-29, wherein one or more of the adjuvants is chosen from gymcitabine, calcium, bleomycin, and cisplatin.

Aspect 31 is the method of any of Aspects 1-30, wherein the adjuvant is capable of increasing or suppressing an immune system response, such as according to a predetermined immune response.

Aspect 32 is the method of any of Aspects 1-31, wherein the adjuvant is capable of increasing a volume of cells killed.

Aspect 33 is the method of any of Aspects 1-32, wherein the adjuvant is capable of changing the cell death mechanism.

Aspect 34 is a method of tissue ablation comprising: applying a series of electrical pulses to a tissue; wherein one or more pulse parameter (e.g., pulse width, repetition rate/frequency, etc.) is modified to manipulate volumes of tissue undergoing different types of cell death; optionally wherein the modifying of the pulse parameter(s) is performed before, during and/or after the applying of the electrical pulses and comprises selecting the pulse parameter(s) to achieve a desired, selected and/or predetermined gradient or pattern of cell death in the tissue.

Aspect 35 is the method of any of Aspects 1-34, wherein one or more cell death mechanism attributable to one or more of the different types of cell death is controlled to elicit a specific, desired and/or predetermined patient-specific treatment outcome, such as by controlling the extent of one or more of the different types of cell death during the applying of the series of electrical pulses.

Aspect 36 is the method of any of Aspects 1-35, wherein: pulse width is reduced to increase the volume of tissue experiencing regulated cell death (RCD), such as apoptosis and/or pyroptosis; and/or pulse repetition rate is increased to elicit a larger volume of necrosis and/or necroptosis; wherein the pulse width reduction or the pulse repetition increase is performed before, during and/or after the applying of the electrical pulses; optionally wherein the pulse width reduction or the pulse repetition increase is measured relative to another pulsing protocol having a known outcome (such as a protocol resulting in less RCD) in order to achieve a different and/or modified outcome (such as more RCD) due to the difference(s).

Aspect 37 is the method of any of Aspects 1-36, wherein: pulse width, interphase delay and/or interpulse delay are modified in a manner capable of adjusting volumes of pyroptosis and/or necroptosis to achieve a desired immunological outcome, such as a predetermined immune response; wherein the pulse width, interphase delay and/or interpulse delay are modified before, during and/or after the applying of the electrical pulses; optionally wherein the pulse width, interphase delay and/or interpulse delay are modified relative to another pulsing protocol having a known outcome (such as a certain extent of pyroptosis and/or necroptosis) in order to achieve a different and/or modified outcome (such as an increase or decrease in the extent of tissue experiencing pyroptosis and/or necroptosis.

Aspect 38 is the method of any of Aspects 1-37, wherein one or more adjuvant (such as adjuvant molecules including calcium) is used before, during and/or after the applying of the electrical pulses to increase necrosis/necroptosis and/or is otherwise introduced to manipulate volumes of tissue experiencing different mechanisms of cell death.

Aspect 39 is the method of any of Aspects 1-38, wherein one or more adjuvant (such as adjuvant molecules including bleomycin and/or cisplatin) is introduced before, during and/or after the applying of the electrical pulses, such as to the tissue or a region of interest thereof) to increase overall volumes of cell death while maintaining and/or minimizing immunological effects.

Aspect 40 is a method for tissue ablation comprising: applying a series of electrical pulses to a tissue using one or more electrode; modifying one or more electrode-based parameter to adjust volumes of cell death (such as increasing necrosis while limiting or reducing coagulative necrosis, or increasing apoptosis while limiting or reducing necrosis); wherein the electrode-based parameter(s) are modified before, during and/or after the applying of the electrical pulses; optionally wherein the electrode-based parameter(s) are modified relative to another pulsing protocol having a known outcome (such as having a certain extent of apoptosis, necrosis and/or coagulative necrosis) in order to achieve a different and/or modified outcome (such as an increase or decrease in the extent of tissue experiencing apoptosis, necrosis and/or coagulative necrosis).

Aspect 41 is the method of any of Aspects 1-40, wherein the modifying comprises modifying one or more of: the number of electrodes, electrode shape and/or other electrode-based parameter; number of insertions, such as a number of necessary insertions; and/or thermal-regulating technologies (e.g., active cooling, phase change materials, endothermic reactions, and/or exothermic reactions).

Aspect 42 is the method of any of Aspects 1-41, wherein the electrical pulses, plurality of electrical pulses, electroporation protocol, pulsing protocol, or electroporation treatment parameter is/are applied with: a positive pulse width of above zero up to 1 ms, such as 100 μs; and/or a negative pulse width of above zero up to 1 ms, such as 100 μs; and/or; an interphase delay of above zero up to 1 s; and/or an interpulse delay of above zero up to 10 s; and/or an inter-burst delay of above zero up to 10 s; and/or no interphase, interpulse and/or inter-burst delay; and/or a number of pulses from 1-5,000; and/or a voltage of 1-10,000 V; and/or a frequency of from 1 Hz to 250 kHz; and/or a total energized time of from the smallest pulse width up to 1 s.

Aspect 43 is the method of any of Aspects 1-42, wherein the electrical pulses or plurality of electrical pulses is/are applied in a manner and/or in an amount sufficient to promote a local and/or systemic anti-tumor immune system response, such as a predetermined immune response.

Aspect 44 is the method of any of Aspects 1-43, wherein the tissue comprises cancer cells and/or non cancer cells.

Aspect 45 is the method of any of Aspects 1-44, wherein the cells are cancer cells.

Aspect 46 is the method of any of Aspects 1-45, wherein the cancer cells are breast, liver, pancreas, prostate, skin, and/or brain cancer cells.

Aspect 47 is the method of any of Aspects 1-46, wherein the electrical pulses or plurality of electrical pulses are delivered in one or more burst(s) of electrical pulses, such as with an inter-burst delay of up to 10 s.

Aspect 48 is the method of any of Aspects 1-47, wherein the electrical pulses are monopolar pulses, bipolar pulses, or a combination thereof.

Aspect 49 is the method of any of Aspects 1-48, wherein the electrical pulses or plurality of electrical pulses is/are capable of one or more of electroporation-based therapy, electroporation, irreversible electroporation, reversible electroporation, electrochemotherapy, electrogenetherapy, supraporation, and/or high frequency irreversible electroporation, or combinations thereof, such as by way of a DC current.

Aspect 50 is the method of any of Aspects 1-49, wherein the electrical pulses are administered from two or more electrodes, and from any number of electrodes, such as 1, 2, 3, 4, 5, 6, 7, 8, 9, 10, 11, 12, 13, 14, 15, 16, 17, 18, 19, or 20 electrodes, and in any configuration relative to one another, such as being delivered by way of one or more pairs of electrodes.

Aspect 51 is the method of any of Aspects 1-50, wherein the electrical pulses or plurality of electrical pulses are delivered at a voltage of 0 V to 10,000 V, such as above 0 V or 1 V up to 10,000 V, and/or from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 5,000 V, up to 7,500 V, or any range in between any of these ranges or endpoints, including as endpoints any number encompassed thereby.

Aspect 52 is the method of any of Aspects 1-51, wherein one or more pulses of the electrical pulses or plurality of electrical pulses have a pulse length in the ns to second range, such as in the nanosecond to ms range, such as from 1 picosecond to 1 ms, or from 1 picosecond to 100 microseconds, or from 1 picosecond to 10 microseconds, or from 1 picosecond to 1 microsecond, or from at least 0.1 microsecond up to 1 second, or from 0.5 microseconds up to 10 microseconds, or up to 20 microseconds, or up to 50 microseconds, such as 15, 25, 30, 35, 40, 55, 60, 75, 80, 90, 100, 110, or 200 microseconds, or any range in between any of these ranges or endpoints, including as endpoints any number encompassed thereby.

Aspect 53 is the method of any of Aspects 1-52, wherein the plurality of electrical pulses has a frequency in the range of 0 Hz to 100 MHz, such as from above 0 Hz or 1 Hz up to 100 MHz, such as from 2 Hz to 100 Hz, or from 3 Hz to 80 Hz, or from 4 Hz to 75 Hz, or from 15 Hz to 80 Hz, or from 20 Hz up to 60 Hz, or from 25 Hz to 33 Hz, or from 30 Hz to 55 Hz, or from 35 Hz to 40 Hz, or from 28 Hz to 52 Hz, or any range in between any of these ranges or endpoints, including as endpoints any number encompassed thereby.

Aspect 54 is the method of any of Aspects 1-53, wherein the electrical pulses have a waveform that is square, triangular, trapezoidal, exponential decay, sawtooth, sinusoidal, and/or alternating polarity.

Aspect 55 is the method of any of Aspects 1-54, wherein a total number of electrical pulses delivered, and/or a total number of pulses delivered per burst, ranges from 1 to 5,000 pulses, such as from at least 1 up to 3,000 pulses, or at least 2 up to 2,000 pulses, or at least 5 up to 1,000 pulses, or at least 10 up to 500 pulses, or from 10 to 100 pulses, such as from 20 to 75 pulses, or from 30 to 50 pulses, such as 1, 5, 10, 15, 20, 25, 30, 35, 40, 45, 50, 60, 70, or 90 pulses, or any range in between any of these ranges or endpoints, including as endpoints any number encompassed thereby.

Aspect 56 is the method of any of Aspects 1-55, further comprising measuring temperature with one or more thermal sensor, such as measuring temperature of the tissue and/or electrodes.

Aspect 57 is the method of any of Aspects 1-56, further comprising measuring impedance and/or conductivity and/or capacitance, such as tissue, intra- or extra-cellular impedance, conductivity and/or capacitance.

Aspect 58 is a system capable of performing any one or more of the methods of Aspects 1-57.

Aspect 59 is a treatment planning system for determining a patient-specific electroporation-based treatment protocol comprising: a processing module operably configured for performing the following stages: receiving and processing information from medical images of a target tissue and preparing a reconstruction model of the target tissue; and using the model of the target tissue and one or more desired cell death mechanism as inputs, constructing one or more protocols each providing a treatment region with parameters for electroporating the target tissue and the volume of the treatment region expected to undergo the desired cell death mechanism(s); and a processor for executing the stages of the processing module.

Aspect 60 is a treatment planning system for determining a patient-specific electroporation-based treatment protocol comprising: a processing module operably configured for performing the following stages: receiving and processing information from medical images of a target tissue and preparing a reconstruction model of the target tissue; using (i) the model of the target tissue, (ii) one or more patient-specific characteristics (such as tissue type (e.g., myocardium, benign neoplastic tissue, malignant neoplasm, etc.), patient immune status, disease severity, comorbidities, and treatment location), and (iii) one or more desired cell death mechanism, constructing one or more protocols each providing a treatment region with parameters for electroporating the target tissue capable of achieving one or more of the desired cell death mechanisms; and a processor for executing the stages of the processing module.

### BRIEF DESCRIPTION OF THE DRAWINGS

The accompanying drawings illustrate certain aspects of implementations of the present disclosure, and should not be construed as limiting. Together with the written description, the drawings serve to explain certain principles of the disclosure.

FIG. 1 is an illustration showing the types of cell death associated with electroporation-based ablative therapy.

FIGS. 2A-B are representative illustrations showing regions of cell death following H-FIRE treatment with a 10-1-10 waveform delivered with solid monopolar probes (FIG. 2A) and for a 2-5-2 waveform delivered with actively cooled probes (FIG. 2B).

FIG. 3A is a schematic illustrating a system for delivering electrical pulses according to an embodiment of the invention.

FIG. 3B is a drawing of a custom concentric cylinder electrode setup according to an embodiment of the invention.

FIG. 3C is a graph showing computed and experimentally obtained changes in temperature over time for different pulse parameters according to embodiments of the invention.

FIG. 3D is a drawing showing electric field and temperature profiles within the hydrogel according to an embodiment of the invention.

FIG. 3E is a graph showing the radial dependence of electric field and temperature for administration of electrical pulses according to an embodiment of the invention.

FIGS. 4A, 4C, and 4E are drawings showing pulse schemes for traditional IRE, including pulse widths of 90 μs (FIG. 4A) and 70 μs (FIG. 4C), and for H-FIRE (FIG. 4E).

FIGS. 4B, 4D, and 4F are drawings illustrating electroporation and cell death outcomes of the pulse parameters shown in FIGS. 4A, 4C, and 4E, respectively.

FIG. 5A is a is a fluorescence microscopy image of live-dead staining of cells treated with various pulse schemes.

FIG. 5B is a graph showing the areas of lesions produced from electroporating cells using various pulse schemes.

FIG. 5C is a graph showing the electric field threshold for several pulse schemes.

FIG. 6A is an electric field distribution map showing the applied electric field between two plate electrodes.

FIG. 6B is a drawing showing the percent difference between the applied electric field shown in FIG. 6A and the intended electric field.

FIG. 6C is an illustration showing the release of DAMPs as detected using a colorimetric assay following electroporation of tumor cells.

FIG. 7A is a graph showing the concentration of extracellular ATP present following electroporation of cells using various pulse schemes.

FIG. 7B is a graph showing the concentration of extracellular ATP present 24 hours following electroporation of cells using various pulse schemes.

FIG. 8A is a graph showing the absorbance of cells that were harvested and lysed after exposure to electroporation using various pulse schemes.

FIG. 8B is a graph showing the increase in caspase 3/7 activity for cells electroporated using a variety of pulse schemes.

FIG. 9A is a graph showing fluorescence measurements of cells electroporated using a variety of pulse schemes.

FIG. 9B is a graph showing the increase in caspase 1 activity for cells electroporated using a variety of pulse schemes.

FIG. 10 is an illustration showing the cell death distribution for an ablation area exposed to electrical pulses from one, two, and three pairs of electrodes.

FIG. 11 is an illustration showing the presence of pro-inflammatory signaling molecules and types of cell death associated with short and long pulses, such as apoptosis, necrosis, pyroptosis, programmed necrosis etc.

### DETAILED DESCRIPTION OF VARIOUS EMBODIMENTS OF THE INVENTION

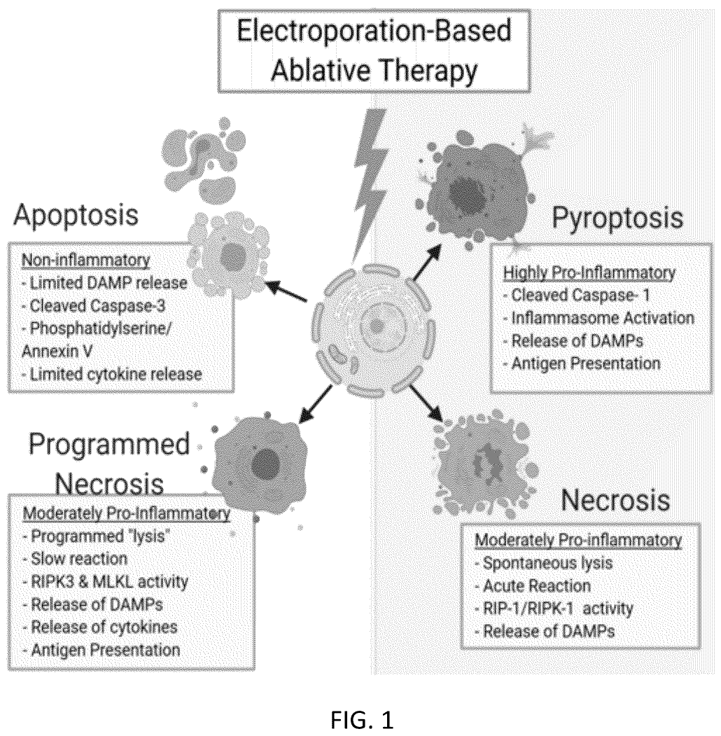

HFIRE uses several pulse parameters that can be customized to create a tailored cell death profile for optimal immune responses. The short bursts of bi-polar electric fields utilized by HFIRE generate nanoscale defects in cell membranes leading to cell death. FIG. 1 shows the characteristics of four types of cell death associated with electroporation-based ablative therapy: apoptosis, pyroptosis, necrosis, and programmed necrosis. While IRE-induced cell death is primarily characterized as apoptosis, pyroptosis has also been identified for H-FIRE treatment as an inflammatory form of cell death that may lead to a more effective local and systemic immune response (Ringel-Scaia, 2019). Furthermore, HFIRE treatment can induce necroptosis (MLKL-dependent cell death) (Wasson, 2020). By tailoring pulse parameters, the mode of cell death, and therefore the type and/or level of immunogenicity, can be tailored.

Unlike IRE, several pulse parameters contribute to a given H-FIRE voltage waveform—positive pulse width, interphase delay, negative pulse width, interpulse delay, energized time, etc. Each provides an opportunity to manipulate clinical outcomes. In embodiments, a plurality of electrical pulses is administered.

In embodiments, one or more of the electrical pulses can be monopolar. In embodiments, one or more of the electrical pulses is bipolar. In embodiments, the electrical pulses are delivered with an AC current, DC current, or a combination of AC and DC currents.

In embodiments, each pulse comprises a positive and/or negative polarity pulse width of up to about 100 μs, such as up to 100 ns, 250 ns, 500 ns, 1 μs, 2 μs, 5 μs, 10 μs, 15 μs, 20 μs, 25 μs, 40 μs, 50 μs, 75 μs, or 100 μs, or any range in between any of these ranges or endpoints.

In embodiments, each of the electrical pulses comprises an interphase delay (a delay between positive and negative polarity portions of a bipolar pulse) of up to about 100 μs, such as up to about 100 ns, 250 ns, 500 ns, 750 ns, 1 μs, 2 μs, 3 μs, 4 μs, 5 μs, 6 μs, 7 μs, 8 μs, 9 μs, 10 μs, 15 μs, 20 μs, 25 μs, 50 μs, or 75 μs, or any range in between any of these ranges or endpoints. In other embodiments, the electrical pulses change polarity instantly or without an interphase delay.

In embodiments, the pulses of the plurality of electrical pulses are separated by an interpulse delay. In embodiments, the plurality of electrical pulses may be administered with no delay, or effectively no delay, between pulses. If administered with an inter-pulse delay, the delay between pulses can be up to 100 times, 50 times, 20 times, 10 times, or 5 times the pulse length, such as 3 times the pulse length, 2 times the pulse length, equal to the pulse length, or less than the pulse length. For example, the delay between pulses can be 10%, 20%, 25%, 40%, 50%, 60%, 75%, 80%, or 90% of the pulse length.

In embodiments, the total pulse length (including positive pulse, interphase delay, negative pulse width, and interpulse delay) is in the nanosecond to microsecond range, including from 1 picosecond to 1 ms, or from below 1 microsecond, or from at least 0.1 microsecond up to 5 microseconds, or from 0.5 microseconds up to 2 microseconds or up to 10 microseconds, such as up to 100 ns, 250 ns, 500 ns, 1 μs, 2 μs, 5 μs, 10 μs, 15 μs, 20 μs, 25 μs, 40 μs, 50 μs, 75 μs, 100 μs, 125 μs, or 150 μs, or even up to about 200 μs or any range in between any of these ranges or endpoints, including as endpoints any number encompassed thereby.

In embodiments, the total energized time is up to about 100 ms, such as up to about 1 μs, 5 μs, 10 μs, 20 μs, 30 μs, 40 μs, 50 μs, 60 μs, 70 μs, 80 μs, 90 μs, 100 μs, 125 μs, 150 μs, 200 μs, 250 μs, 500 μs, 1 ms, 2 ms, 5 ms, 10 ms, 25 ms, 50 ms, or 75 ms.

The X-X-X convention referred to in this disclosure can mean any one or more of a pulsing protocol of the following formats: pulse ontime-pulse offtime-pulse ontime; positive pulse-delay/offtime-negative pulse; negative pulse-delay/offtime-positive pulse; positive portion of a pulse-delay/offtime-negative portion of pulse, etc. IRE pulses were administered as monopolar pulses at a frequency of one pulse per second. In embodiments, the plurality of electrical pulses can have a pulsing scheme that incorporates one or more inter-burst delays, such as pulsing schemes of bursts of pulses comprising schemes of 1-1-1 μs, 2-1-2 μs, 5-1-5 μs, or 10-1-10 μs with up to a 1-second delay between bursts.

In embodiments, the total number of pulses delivered or the number of pulses per burst ranges from 1 to about 5,000 pulses, such as from at least 1 up to 3,000 pulses, or at least 2 up to 2,000 pulses, or at least 5 up to 1,000 pulses, or at least 10 up to 500 pulses, or from 10 to 100 pulses, such as from 20 to 75 pulses, or from 30 to 50 pulses, such as 1, 5, 10, 15, 20, 25, 30, 35, 40, 45, 50, 60, 70, or 90 pulses.

In embodiments a single burst of electrical pulses is delivered. In other embodiments, multiple bursts are delivered, each comprising up to about 100 pulses, such as up to about 5, 10, 15, 20, 25, 30, 40, 50, 60, 70, 80, or 90 pulses.

In embodiments, the pulses or bursts are delivered at a frequency in the range of about 0 Hz to 100 MHz, such as from above 0 Hz or 1 Hz up to 100 MHz, such as from 2 Hz to 100 Hz, or from 3 Hz to 80 Hz, or from 4 Hz to 75 Hz, or from 15 Hz to 80 Hz, or from 20 Hz to 60 Hz, or from 25 Hz to 33 Hz, or from 30 Hz to 55 Hz, or from 35 Hz to 40 Hz, or from 28 Hz to 52 Hz, or a frequency ranging from about 100 Hz to 100 MHz, such as in the Hz range from 100 Hz or 1 Hz up to 100 Hz, or from 2 Hz to 100 Hz, or from 3 Hz to 80 Hz, or from 4 Hz to 75 Hz, or from 15 Hz to 80 Hz, or from 20 Hz to 60 Hz, or from 25 Hz to 33 Hz, or from 30 Hz to 55 Hz, or from 35 Hz to 40 Hz, or from 28 Hz to 52 Hz, or a frequency in the kHz or MHz range, such as from 1 kHz to 10 kHz, or from 2 kHz to 8 kHz, or from 3 kHz to 5 kHz, or from 4 kHz to 9 kHz, or from 7 kHz to 15 kHz, or from 6 kHz to 20 kHz, or from 12 kHz to 30 kHz, or from 25 kHz to 40 kHz, or from 5 kHz to 55 kHz, or from 50 kHz to 2 MHz, including any range in between, such as from 10 kHz to 25 kHz, or from 15 kHz to 40 kHz, or from 20 kHz to 50 kHz, or from 75 kHz to 150 kHz, or from 100 kHz to 175 kHz, or from 200 kHz to 250 kHz, or from 225 kHz to 500 kHz, or from 1 Hz to 400 kHz, or from 250 kHz to 750 kHz, or from 500 kHz to 1 MHz, or any range in between any of these ranges or endpoints, including as endpoints any number encompassed thereby.

In embodiments of the invention, the plurality of electrical pulses are administered at a voltage in the range of 0 V to 10,000 V, such as above 0 V or 1 V up to 10,000 V, and/or from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 5,000 V, up to 7,500 V, or for example from 100 V to 15,000 V, such as from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 15,000 V, up to 7,500 V, from 4,000 V to 12,000 V, such as less than 450 V, or less than 425 V, such as from above 0 V to 400 V, or from about 10 V to 350 V, or about 20 V to about 300 V, or about 30 V to about 250 V, or from about 15 V to about 200 V, or from about 50 V to about 150 V, or about 75 V to 100 V, or from 30 V to 225 V, or from 60 V to 375 V.

In embodiments, the shape of the electrical pulses delivered can be any desired waveform, including square, triangular, trapezoidal, exponential decay, sawtooth, sinusoidal, and/or such waveforms comprising one or more pulses of alternating polarity.

In embodiments, the electrical pulses are administered from two or more electrodes, and from any number of electrodes, such as 1, 2, 3, 4, 5, 6, 7, 8, 9, 10, 11, 12, 13, 14, 15, 16, 17, 18, 19, or 20 electrodes, and in any configuration relative to one another, such as being delivered by way of one or more pairs of electrodes.

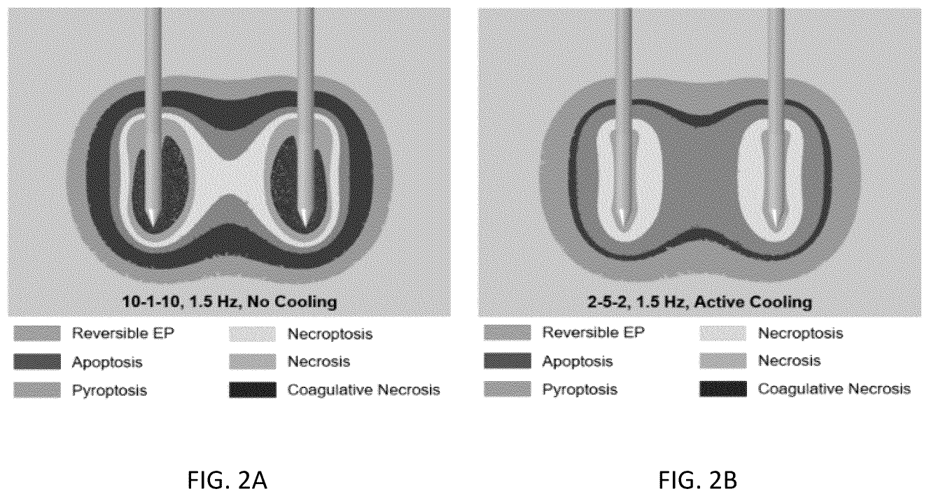

In embodiments, the gradient of cell death can be tailored using active cooling. FIG. 2A shows representative regions of cell death following treatment with a 10-1-10 waveform using solid monopolar probes. FIG. 2B shows representative regions of cell death following treatment with a 2-5-2 waveform with actively cooled probes.

The following Example is illustrative and should not be interpreted to limit the scope of the claimed subject matter.

### Examples

As cell death mechanisms can vary based on the pulsing parameters and the electroporation-induced immune response is a direct outcome of the cell death pathway elicited, different degrees of immunogenicity and immune cell infiltration can be selected with different pulsing protocols. Embodiments of the present invention include methods directed to selecting pulsing protocols to achieve a desired cell death mechanism and immune response. In embodiments, a user can choose to elicit specific cell death outcomes depending upon the application and desired dynamics of immune infiltration after treatment. For example, in most oncological applications, clinicians will seek to employ treatment parameters that yield the strongest immune stimulation.

Bursts of pulses with pulse durations of ˜5 μs are likely to achieve strong immune stimulation with the highest likelihood of success. To preserve strong immunogenicity, treatment voltage, repetition rate, and probe design can be tuned to ensure that tissue is not thermally damaged. Bursts employing shorter pulse durations are less efficient in terms of ablating tissue, so high voltages are needed to achieve targeted ablation sizes. Waveforms with longer pulse durations do not appear to be as effective at stimulating cytotoxic T cells. Monopolar pulsing schemes are known to elicit strong electrochemical effects near the electrodes, which could disrupt or enhance the targeted volume of cell death, as well as the distribution of each cell death pathway. The above considerations will aid in the selection of the precise pulse duration, interphase delay, and interpulse delay that yield the desired pro-inflammatory cell death mechanisms.

In some applications, targeted ablation volumes (or depths) may be desired along with less immune stimulation. For instance, in cardiac treatments, acute post-treatment inflammation is associated with increased risk of arrhythmogenicity and recurrence of atrial fibrillation (Andrade, J. G. et al., “Early recurrence of atrial tachyarrhythmias following radiofrequency catheter ablation of atrial fibrillation”, PACE—Pacing Clin. Electrophysiol. 35, 106-116, 2012). In these treatments, waveforms capable of inducing “quiet” cell death may be more favorable. Bipolar burst waveforms with very short pulse durations (i.e. ˜1 μs) are likely optimal. Maintaining a short interphase delay will likely be necessary to mitigate cardiomyocyte stimulation. To further mitigate any inflammation, the interpulse delay can also be extended, which could reduce the severity of electrically induced pressure and mitigate microbubble formation. Finally, incorporating irrigation within the probe core to maintain tissue temperature may also be desired to allow for maximum control of downstream effects. These “immune silent” ablations may also be favored in other contexts such as for other endovascular applications, or in patients with existing autoimmune disorders.

Both non- and pro-inflammatory cell death mechanisms are associated with positive outcomes depending upon various factors (Brock, R. M. et al., “Starting a Fire Without Flame: The Induction of Cell Death and Inflammation in Electroporation-Based Tumor Ablation Strategies”, Front. Oncol. 10, 2020). As stated above, IRE pulse parameters (Table 1) play a major role in the type of cell death initiated, and the tissue volume experiencing a given cell death mechanism affects immunogenicity. These pulse parameters—along with the chosen geometric configuration—can be tuned to generate a predetermined distribution of different cell death mechanisms. The preferred cell death mechanisms may depend upon but are not limited to: tissue type (e.g., myocardium, benign neoplastic tissue, malignant neoplasm, etc.), patient immune status and overall health, disease severity, comorbidities, and/or treatment location. Manipulation of electrode and treatment parameters will dictate the degree of acute and delayed inflammation. The extent of innate and adaptive immunity can be controlled to generate the optimal immune profile given each patient's unique case.

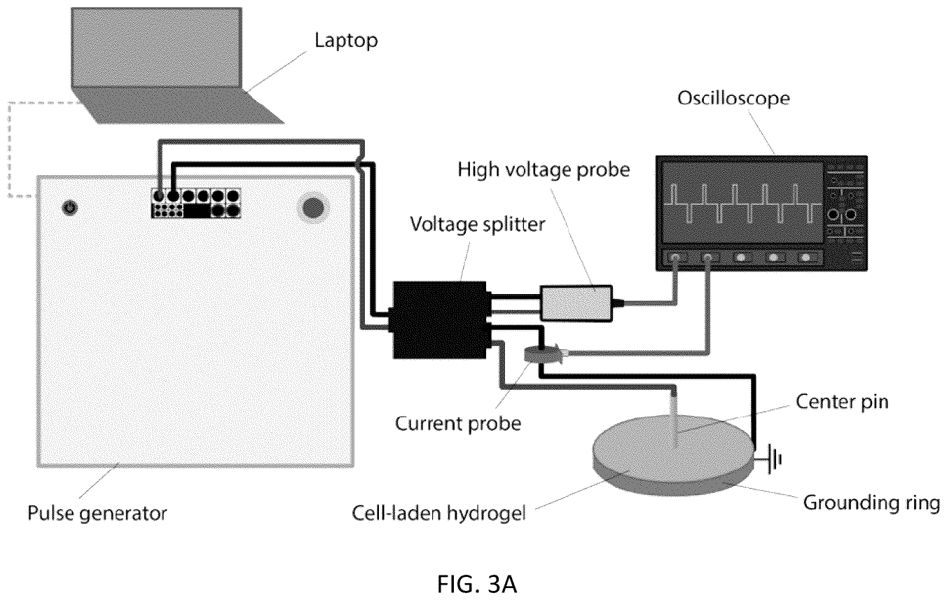

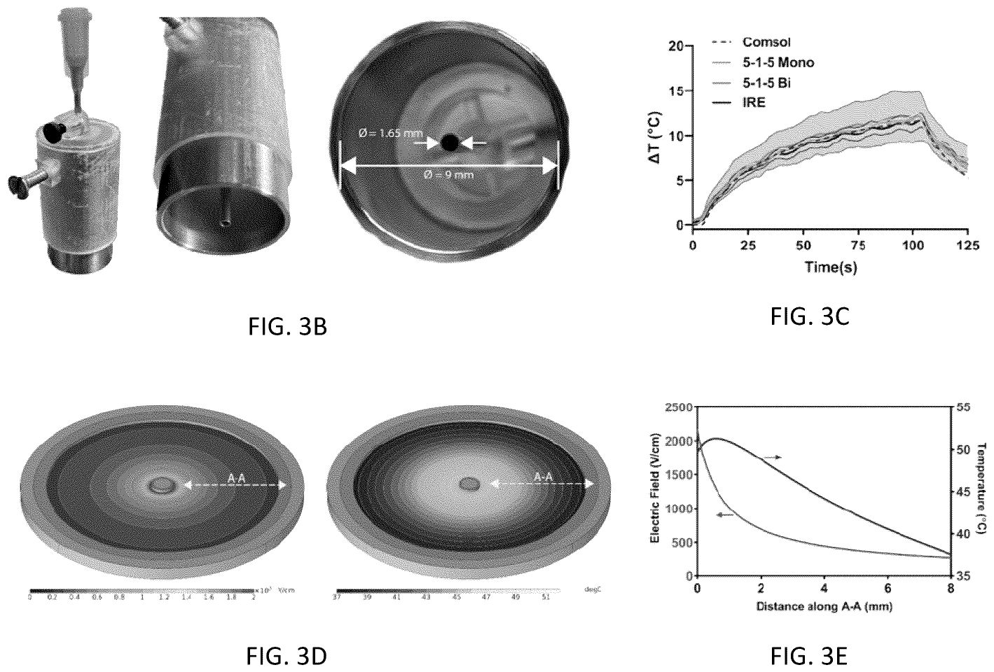

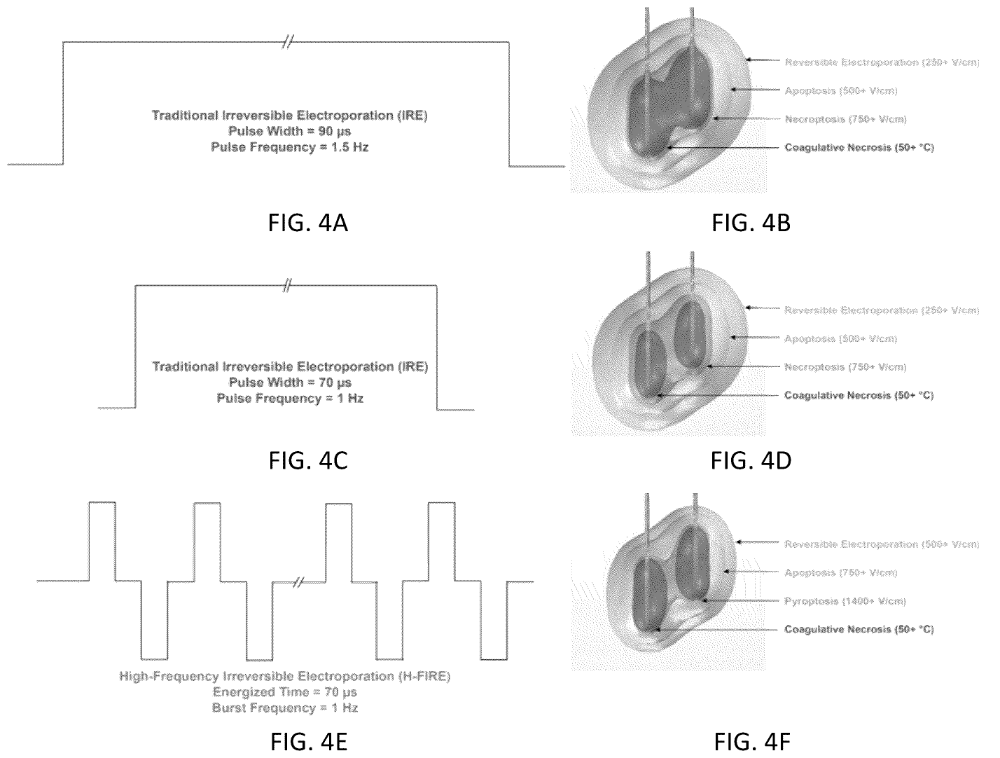

As shown in Table 1, certain pulse parameters and adjuvant molecules will have unique effects in terms of which type of cell death is generated at different electric field strengths. To quantitatively characterize these effects, a series of in vitro experiments were carried out. Different cell types were cultured and treated. The hallmark characteristics of each cell death mechanism (listed in Table 2) were measured at various electric field magnitudes. The specified electrode configuration generates an exponential electric field gradient with the highest fields near the center electrode and the lowest at a concentric outer ring electrode (FIGS. 3A-E). This setup provides a simple relationship between the spatial location of the cells within the platform and the electric field strength they are exposed to. This allows for the prediction of cell death volumes in clinical treatments, as illustrated schematically in FIGS. 4A-F.

TABLE 1
PEF parameters that can be changed to modulate cell death mechanisms

| | | | |
|---|---|---|---|
| | | Effect on | Anticipated effect on |
| Parameter | Effect on Pores | Heat | Immune System |

Pulse Parameters (range)

| | | | |
|---|---|---|---|
| Pulse Width (0.2 μs-1 ms) | Increases with | None | Longer -> more necrotic |
| | longer pulses | | |
| Interphase Delay (0.2- | Depends on | Negligible | Unknown |
| 1,000 μs) | waveform Polarity | | |
| Interpulse Delay | Negligible | Negligible | Unknown |
| (0.2-1,000 μs) | | | |
| Energized Time | Increases but | Increases | Longer -> more necrotic |
| (1-1,000 μs/s) | limited | | |
| Burst Number | Increases | Increases | Higher -> more necrotic |
| (1-400) | | | |
| Burst Frequency | unknown | Increases | Higher -> more necrotic |
| (0.1-10 Hz) | | | |
| Voltage | Increases | Increases | Higher -> more necrotic |
| (10-5,000 V) | | | |
| Polarity | Unipolar increases | None | Unipolar -> more necrotic |
| (+, −, combination) | | | |

Adjuvants

| | | | |
|---|---|---|---|
| Calcium | Increases | N/A | More Ca²⁺ -> more necrotic |
| Bleomycin | None | NA | None, but increases volume |
| | | | of cell death |
| Cisplatin | None | N/A | Immunosuppressive |

TABLE 2
Types of Cell Death and Molecules that can be used to measure them in vitro

| Cell Death | | Signaling Pathways | |
|---|---|---|---|
| Type | Immunogenicity | Implicated | Measurement Strategies |
| Apoptosis | Lowest | Caspase 3/7 | Fluorometric assays |
| Pyroptosis | High | Caspase 1 | Fluorometric assays |
| Necroptosis | Moderate | RIPK1, RIPK3, MLKL | Western blot |
| Necrosis | Highest | RIP1, PARP | Fluorometric assays and |
| | | | ELISAs to measure ATP, |
| | | | HMGB1 |

### Cell Culture

Human hepatocellular carcinoma (HepG2, ATCC HB-8065, Manassas, VA) cells were cultured in Eagle's Minimum Essential Medium (EMVIEM, ATCC) and human glioblastoma (U-251, Sigma-Aldrich 09063001, St. Louis, MO) cells were cultured in Dulbecco's Modified Eagle Medium (DMEM, ATCC); each was supplemented with 10% FBS (Fisher Scientific, Hampton, NH) and 1% penicillin-streptomycin (Fisher Scientific). Cells were incubated at 37° C. with 5% CO₂ and sub-cultured regularly at ˜80% confluency.

When confluent, cells were detached from flasks with 0.25% trypsin-EDTA (Fisher Scientific) and seeded as a monolayer cultured in a 12-well plate. Plates were incubated overnight at 37° C. to allow for cell attachment and adaptation of natural morphology.

### Pulsed Electric Field Treatment

Approximately 24 hours after seeding, media was removed from scaffolds. A low conductivity buffer (containing HEPES, CaCl₂ and sucrose) was added on top of the monolayer immediately prior to pulsing. A custom-made ring and pin electrode (Øₚᵢₙ=1.85 mm; Ø_{ring}=18 mm) was inserted into each well. Bursts were applied with a high-voltage generator (VoltMed Inc., Blacksburg, VA). Waveforms were adjusted to achieve the desired energized time and bursts were applied at a specified frequency. Pre-warmed culture media was replenished immediately after treatment.

### Quantifying the Modes of Cell Death

The extent of inflammatory cell death was quantified through measuring the release of DAMPs such as ATP and HMGB1. To quantify what mode of immunologic cell death is dominant in each group, Caspase 3, Caspase 1 and MLKL activation were measured as metrics for apoptosis, pyroptosis, and necroptosis, respectively. Caspase 1 activation was detected with a fluorometric assay while MLKL activation was measured using western blot analysis. When appropriate, cells were lysed using RIPA buffer. The lysate was then studied for the marker of interest.

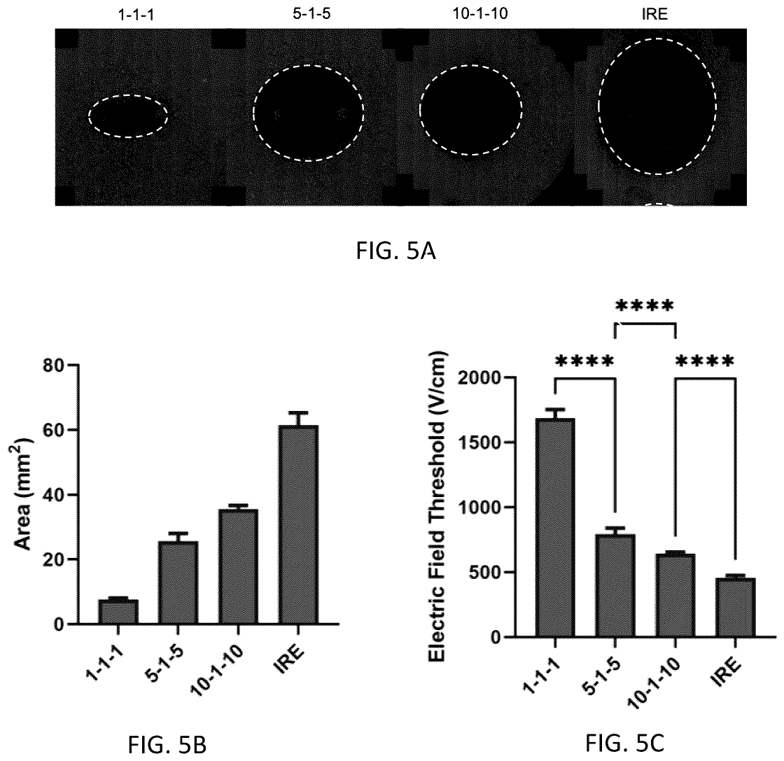

The monolayer of cells was treated with a non-uniform electric field and the ablation areas were measured 24 hours after the treatment. The live and dead areas were visualized through fluorescent microscopy (FIG. 5A). The ablation areas were measured using Image J and correlated to corresponding electric fields characterized through a model in comsol (FIG. 5B). Electric field thresholds were used to normalize the applied field among waveforms. FIG. 5C depicts the electric field thresholds for all applied waveforms. Cell death is expected when exposed to fields equal to or above the thresholds.

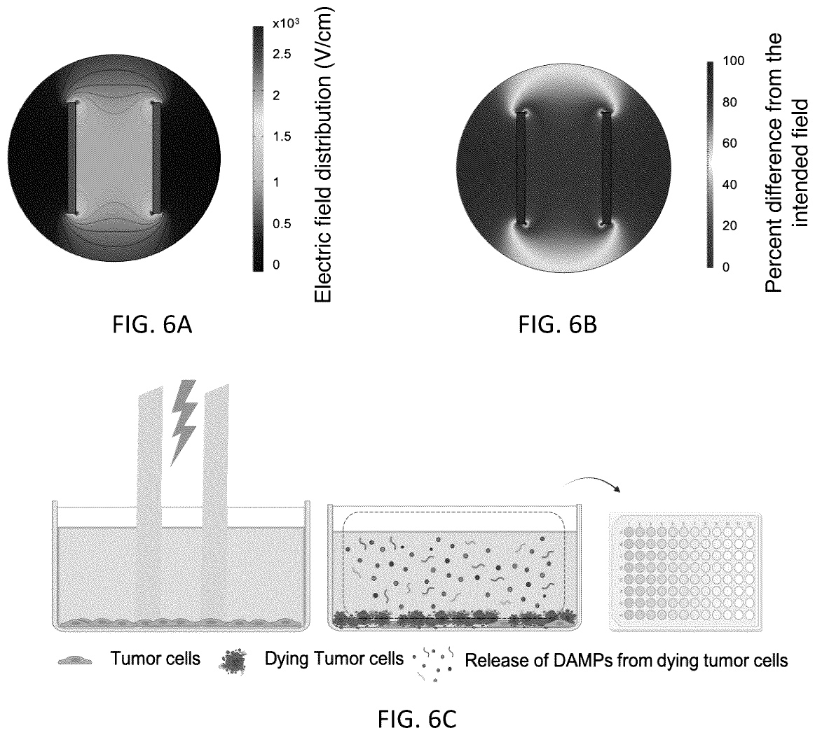

Next, these electric field thresholds were used to apply four different waveforms (1-1-1, 5-1-5, 10-1-10, and IRE) at various electric fields (0.5, 1, 1.5, and 2 times the electric field threshold) while having comparable levels of death. Plate electrodes were used to apply relatively uniform electric fields (FIGS. 6A-B). The supernatant was analyzed to determine the extent of the release of damage associated molecular patterns (DAMPs) (FIG. 6C). DAMPs are proteins that can start a signaling cascade that would lead to activation of both innate and adaptive immune response.

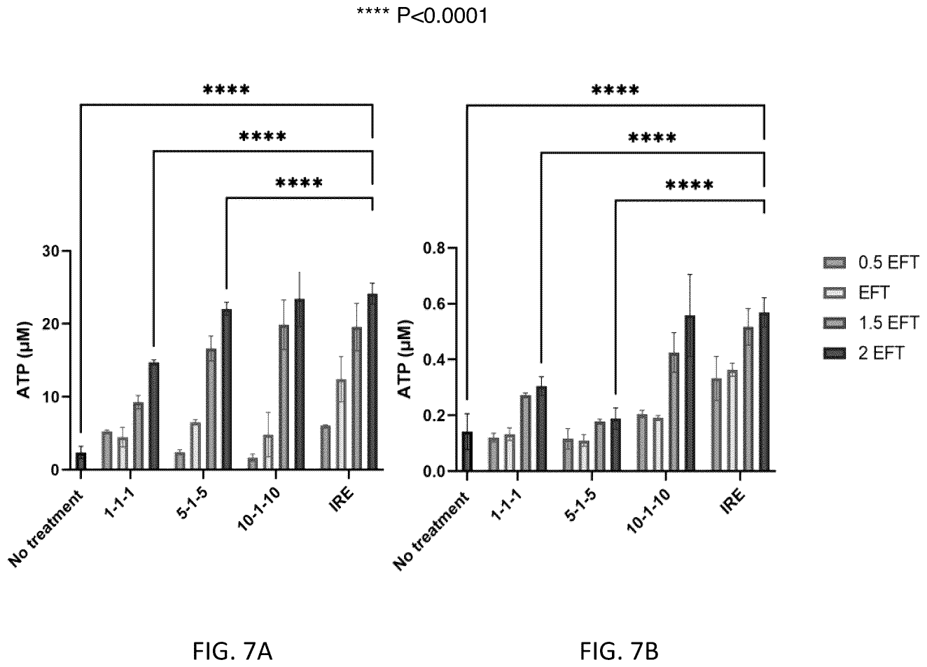

In particular, the release of ATP could directly correlate to levels of inflammation. The level of ATP in the supernatant was measured as a representative of release of DAMPs leading to increased inflammation (FIGS. 7A-B). An ATP assay was performed for several waveforms (1-1-1, 5-1-5, 10-1-10, and IRE) and fields (0.5, 1, 1.5, and 2 times the electric field threshold). The applied electric fields were adjusted to achieve the same level of cell death across the various pulse parameters (lower voltages were applied for longer pulses). It was observed that the level of ATP release is field dependent. The majority of the ATP is released from the treated cells within half an hour of treatment (FIG. 7A). At 24 hours, some ATP release was still observed but at much lower amounts (FIG. 7B). It was also observed that ATP release was increased when longer pulses were applied. Furthermore, the concentration of ATP was significantly higher when the applied electric field was increased to double the measured field thresholds. These results suggest that the inflammatory response can vary based on the applied field as well as the pulse width.

Caspase 3/7 has been used to identify apoptosis. Cells treated with IRE and HFIRE show differences in caspase activation. Time dependent studies show the highest levels of caspase activation at 6 hours (Mercadal, 2020).

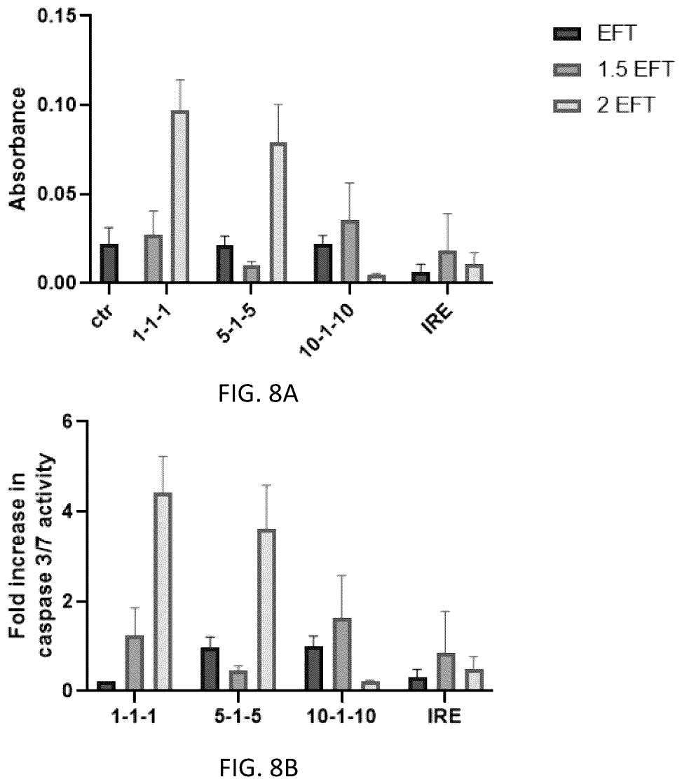

Here, apoptosis levels were measured through caspase 3/7 activity at the electric field threshold and at 1.5 and 2 times the electric field threshold. Cells were harvested at 6 hours and lysed. Samples were adjusted to have the same levels of protein. Caspase activation was measured using a colorimetric assay (FIG. 8A-B). An increase in active caspase 3 concentration was found for shorter pulses, and with increases in electric field. Not much caspase activity was observed in the IRE and 10-1-10 groups.

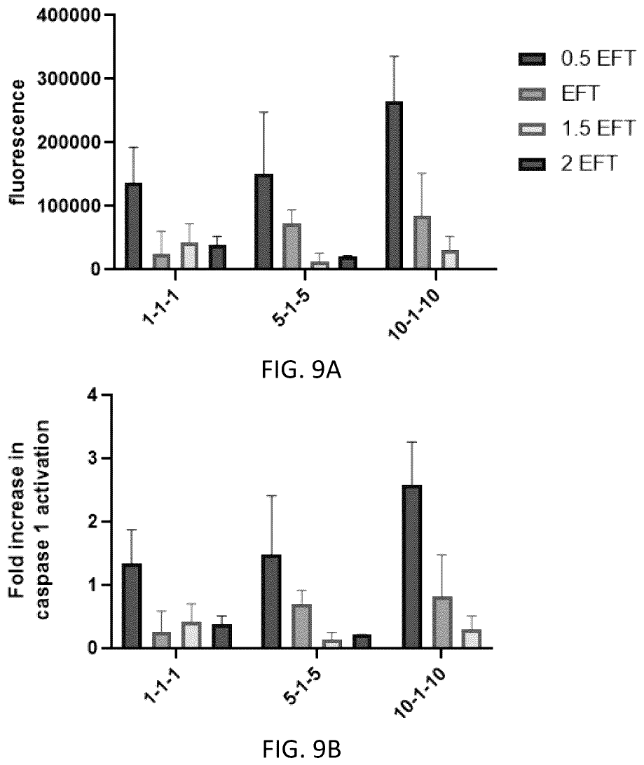

Caspase 1 activity was also measured as it is indicative of pyroptosis. Cells were harvested at 0.5 hours, samples were adjusted to have the same levels of protein, and caspase activation was measured using a fluorometric assay (FIGS. 9A-B). An increase in caspase 1 concentration was observed in lower sublethal fields. As the field was increased, the caspase activity decreased. The trends observed for caspase 1 are the opposite of caspase 3/7.

Based on these results, treatment parameters (i.e. pulse width, the applied field, inter-pulse delay, electrode orientation, cooling strategies, etc.) can be controlled to affect cell death outcomes, type(s) of immune response, (for example, pyroptosis is highly inflammatory whereas apoptosis is not), and/or the level of inflammatory response (from non-inflammatory to highly inflammatory), based on individuals' specific needs.

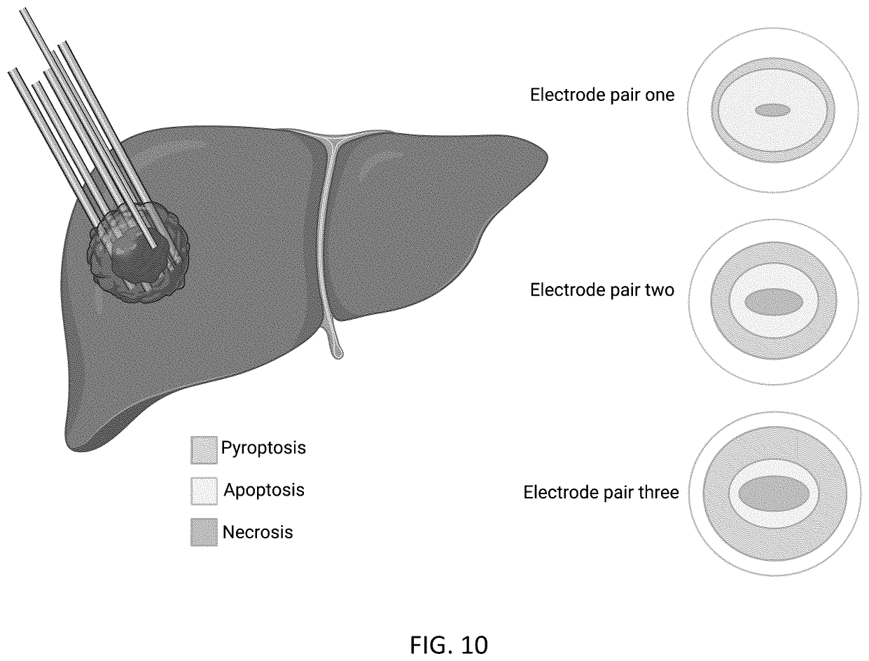

Further, a multi electrode array can be utilized to obtain various forms of cell death response in different areas of the target. As shown in FIG. 10, a multi electrode array can be used to tailor the type of cell death to favor pyroptosis, apoptosis, or necrosis. It can also be envisioned that one pair of electrodes could be used to apply longer low magnitude pulses to achieve one form of cell death (i.e. pyroptosis) and another pair used to achieve other form of cell death (i.e. apoptosis).

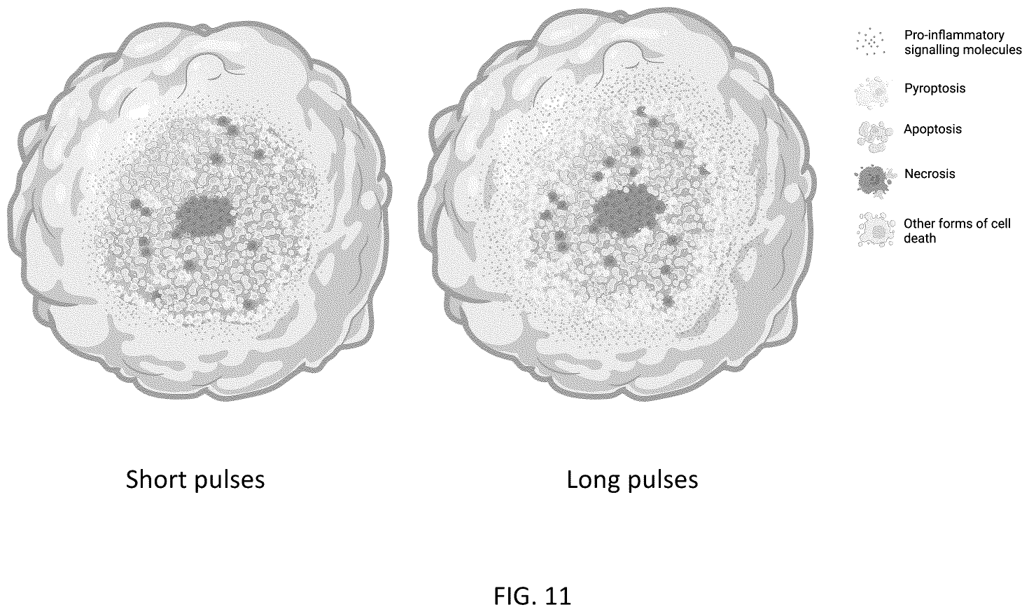

FIG. 11 is an illustration of an example cell death distribution and pro-inflammatory signaling molecule concentration for short and long pulses. As demonstrated in FIGS. 8A-9B, the dominant cell death mechanism is expected to shift when altering the pulse width. As a result, higher levels of apoptosis are expected to be obtained when shorter pulses are applied. On the contrary, increased levels of pyroptosis are expected to be observed with longer pulses. Correspondingly, higher levels of DAMPs (e.g. ATP, HMGB1, etc.) and proinflammatory mediators such as IL-1β or IL-18 are expected to be released when applying longer pulses.

Any method described herein can be embodied in software or set of computer executable instructions capable of being run on a computing device or devices. The computing device or devices can include one or more processor (CPU) and a computer memory. The computer memory can be or include a non-transitory computer storage media such as RAM which stores the set of computer-executable (also known herein as computer readable) instructions (software) for instructing the processor(s) to carry out any of the algorithms, methods, or routines described in this disclosure. As used in the context of this disclosure, a non-transitory computer readable medium (or media) can include any kind of computer memory, including magnetic storage media, optical storage media, nonvolatile memory storage media, and volatile memory. Non-limiting examples of non-transitory computer-readable storage media include floppy disks, magnetic tape, conventional hard disks, CD-ROM, DVD-ROM, BLU-RAY, Flash ROM, memory cards, optical drives, solid state drives, flash drives, erasable programmable read only memory (EPROM), electrically erasable programmable read-only memory (EEPROM), non-volatile ROM, and RAM. The computer-readable instructions can be programmed in any suitable programming language, including JavaScript, C, C#, C++, Java, Python, Perl, Ruby, Swift, Visual Basic, and Objective C. Embodiments of the invention also include a non-transitory computer readable storage medium having any of the computer-executable instructions described herein.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compositions disclosed herein or with any other compositions. Likewise, any of the disclosed compositions can be used with any of the methods disclosed herein or with any other methods. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A method of treating tissue with electrical pulses comprising:
administering one or more or a plurality of electrical pulses to cells of a target tissue according to a pulsing protocol expected to achieve a desired cell death gradient based on one or more cell death mechanism chosen from apoptosis, pyroptosis and/or necroptosis.

2. (canceled)

3. The method of claim 1, wherein the cell death gradient is capable of:
shifting a local tumor microenvironment from anti-inflammatory to pro-inflammatory, or from pro-inflammatory to anti-inflammatory, in response to the administering of the plurality of electrical pulses; or
activating a local innate immune response and a systemic anti-tumor adaptive immune response, reducing metastatic lesions and/or preventing recurrence in tumors in response to the administering of the plurality of electrical pulses.

4-6. (canceled)

7. The method of claim 1, further comprising monitoring:
the extent of one or more of the cell death(s) mechanisms elicited.

8. The method of claim 1, further comprising adjusting one or more parameter of the pulsing protocol to change the gradient and/or extent of cell death in response to the monitoring.

9. (canceled)

10. The method of claim 1, wherein the pulsing protocol is constructed based on reference to a model.

11-13. (canceled)

14. The method of claim 10, wherein
the model is prepared by receiving and processing information from medical images of the target tissue and preparing a reconstruction model of the target tissue.

15. (canceled)

16. The method of claim 1, further comprising:
adjusting one or more parameter of the electroporation protocol to change the desired cell death mechanism to a different desired cell death mechanism chosen from necroptosis, pyroptosis, or apoptosis; and
applying the electroporation protocol to the cells of the target tissue to elicit the different desired cell death mechanism in the target region.

17-19. (canceled)

20. The method of claim 16, wherein the one or more parameter of the electroporation protocol comprises a parameter chosen from pulse width, voltage, polarity, interpulse delay, interphase delay, frequency, number of pulses, and total energized time.

21-35. (canceled)

36. The method of claim 6, wherein:
pulse width is reduced to increase the volume of tissue experiencing regulated cell death (RCD), such as apoptosis and/or pyroptosis; and/or
pulse repetition rate is increased to elicit a larger volume of necroptosis;
wherein the pulse width reduction or the pulse repetition increase is performed before, during and/or after the applying of the electrical pulses.

37-41. (canceled)

42. The method of claim 1, wherein the plurality of electrical pulses is applied with:
a positive pulse width of above zero up to 100 μs; and/or
a negative pulse width of above zero up to 100 μs; and/or
an interphase delay of above zero up to 1 s; and/or
an interpulse delay of above zero up to 10 s; and/or
an inter-burst delay of above zero up to 10 s; and/or
no interphase, interpulse and/or inter-burst delay; and/or
a number of pulses from 1-5,000; and/or
a voltage of 1-10,000 V; and/or
a frequency of from 1 Hz to 250 kHz; and/or
a total energized time of from the smallest pulse width up to 1 s.

43-44. (canceled)

45. The method of claim 1, wherein the cells are cancer cells.

46-47. (canceled)

48. The method of claim 1, wherein the plurality of electrical pulses comprises monopolar pulses, bipolar pulses, or a combination thereof.

49. The method of claim 1, wherein the plurality of electrical pulses is capable of one or more of electroporation based therapy, electroporation, irreversible electroporation, reversible electroporation, electrochemotherapy, electrogenetherapy, supraporation, and/or high frequency irreversible electroporation, or combinations thereof.

50. (canceled)

51. The method of claim 1, wherein the plurality of electrical pulses is delivered at a voltage of from 500 V up to 5,000 V.

52. The method of claim 1, wherein one or more pulses of the plurality of electrical pulses has a pulse length of from 1 picosecond to 100 microseconds.

53. The method of claim 1, wherein the plurality of electrical pulses has a frequency in the range of 0 Hz to 100 MHz.

54-58. (canceled)

59. A treatment planning system for determining a patient-specific electroporation-based treatment protocol comprising:
a processing module operably configured for performing the following stages:
receiving and processing information from medical images of a target tissue and preparing a reconstruction model of the target tissue; and
using the model of the target tissue, constructing one or more protocols each providing parameters for electroporating the target tissue to achieve a desired cell death gradient based on one or more cell death mechanism chosen from apoptosis, pyroptosis and/or necroptosis; and
a processor for executing the stages of the processing module.

60. (canceled)

61. A method of treating tissue comprising:
administering one or more electrical pulses to a first spatial region of a tissue by way of a first electrode configuration in a manner to achieve apoptosis, pyroptosis and/or necroptosis in a first desired/predetermined cell death volume and/or apoptosis, pyroptosis and/or necroptosis and a first immune system response thereto; and
administering one or more electrical pulses to a second spatial region of the tissue by way of a second electrode configuration in a manner to achieve apoptosis, pyroptosis and/or necroptosis in a second desired/predetermined cell death volume different from the first volume and/or apoptosis, pyroptosis and/or necroptosis and a second immune system response different from the first immune response.

62-63. (canceled)

64. The method of claim 61, wherein the electrical pulses cause irreversible electroporation (IRE) of the tissue.
